(19) United States
(12) Patent Application Publication
(10) Pub. No.: US 2010/0210941 A1
(43) Pub. Date: Aug. 19, 2010

(54) AUTO EXAMINATION SYSTEM FOR INTERVERTEBRAL DISCS

(76) Inventors: Hyoung-Ihl KIM, Gimje-si (KR); Dong-Ah SHIN, Seoul (KR)

Correspondence Address: CANTOR COLBURN, LLP, 20 Church Street, 22nd Floor, Hartford, CT, 06103, US

(21) Appl. No.: 12/705,213

(22) Filed: Feb. 12, 2010

(30) Foreign Application Priority Data
Feb. 16, 2009 (KR) 10-2009-0012465

(51) Int. Cl. A61B 6/00; A61B 5/00

(52) U.S. Cl. 600/431; 600/561

(57) ABSTRACT

Disclosed is an auto examination system for intervertebral discs which injects a contrast material into a human intervertebral disc at a regular velocity through a needle and then measures internal pressure of the intervertebral disc from the outside, and uses data, obtained by synchronizing the measured pressure data and a captured image of the intervertebral disc, as diagnosis data, so as to more accurately identify a pain region of intervertebral discs of a patient based on a variation in the internal pressure of the intervertebral disc.

### BACKGROUND OF THE INVENTION

1. Field of the Invention

The present invention relates to an auto examination system for intervertebral discs, and more particularly to an auto examination system for intervertebral discs which injects a contrast material into a human intervertebral disc at a regular velocity and simultaneously measures internal pressure of the intervertebral disc from the outside, and uses data, obtained by synchronizing the measured pressure data and a captured image of the intervertebral disc, as diagnosis data, so as to more accurately identify a pain region of intervertebral discs of a patient based on a variation of the internal pressure of the intervertebral disc through the moving image and the measured pressure data.

2. Description of the Related Art

In general, an auto examination system for intervertebral discs is an apparatus based upon improvements in conventional discography, which examines a degree of degenerative change of an intervertebral disc of a patient, and gradually increases internal pressure of the intervertebral disc through a contrast material injected into the intervertebral disc to induce pain and thus to investigate a relationship between degenerative change and pain of the patient.

It is known that lumbago is a common disease after headaches in that the probability of a person suffering from lumbago during his/her lifetime is approximately 30%. The rate of the lumbago due to intervertebral disc disorder is approximately 15˜40%. Further, it is known that, among methods of testing for intervertebral discs disorder, magnetic resonance imaging (MRI) is most commonly used.

However, using MRI to diagnose ordinary people without lumbago yields an intervertebral disc disorder false-positive rate of approximately 70%. This means that intervertebral disc disorder is not always a cause of lumbago. To put it the other way, although it is found that that a person suffering from lumbago has intervertebral disc disorder as a result of MRI, it is doubtful that intervertebral disc disorder is the cause of lumbago.

Discography is a comparatively old-fashioned examination method which was initially developed in 1948. Discography was considerably deteriorated in value as a high-resolution imaging diagnosis method, but is again gaining popularity as a diagnosis method to induce pain and obtain image information simultaneously as reports on that anatomic disorder may be irrelevant to back pain, revealed by the imaging diagnosis, increases. However, differing from the conventional discography for determining only intervertebral disc disorder, current discography is recreated as a method to judge whether or not pain is induced while gradually increasing internal pressure of intervertebral discs, and simultaneously to evaluate degrees of deformation of the intervertebral discs so as to most accurately identify an intervertebral disc relevant to pain physiologically.

An examination process using the conventional discography will be described as follows. First, a patient lays face down on a bed, and the patient's back is disinfected. Then, an examiner sticks a 22 gauge needle having a length of 5 inches into the patient's back at an angle of 45 degrees through fluoroscopy such that the tip of the needle is located at the center of an intervertebral disc of the patient.

Thereafter, a syringe having a volume of 3˜5 cc is filled with a contrast material (which is radiopaque and thus appears white under fluoroscopy), and is connected to the needle inserted into the patient's back.

The examiner confirms whether or not the patient feels pain while slowly injecting the contrast material through the syringe into the intervertebral disc by hand and observing dispersion of the contrast material within the intervertebral disc through fluoroscopy. When the intervertebral disc is filled with the contrast material and thus the internal pressure of the intervertebral disc is raised, nociceptive nerves distributed in the intervertebral disc are stimulated and thus pain is induced. If the lumbago induced during examination is identical to that which the patient normally feels, it is judged that the intervertebral disc on which the examination is performed is turned out to be positive and thus is a cause of the lumbago.

The inside of the intervertebral disc is filled with nucleus pulposus which mainly consists of proteoglycan and collagen and contains moisture. Nucleus pulposus is in a semi-solid state. Since the liquid contrast material is injected into the semi-solid nucleus pulposus, it takes a considerable time to uniformly disperse the contrast material in the nucleus pulposus. Therefore, if the injection velocity of the contrast material is increased, pressure higher than a normal state is formed, and if the injection velocity of the contrast material is decreased, pressure lower than the normal state is formed. Accordingly, it is necessary to provide a uniform injection velocity of the contrast material.

Conventional discography requires that the examiner operate the syringe filled with the contrast material by hand, thus causing the injection velocity of the contrast material to be non-uniform.

Further, the conventional discography causes the examiner to evaluate the intensity of the internal pressure of the intervertebral disc through pressure transmitted to an examiner's hand or a manometer, thereby increasing a pressure difference due to a contrast material injection velocity difference based on operation of the syringe by the examiner as well as lowering accuracy in pressure measurement.

Moreover, the conventional discography is performed in question and answer form in which the examiner questions whether or not the patient feels pain and the patient answers the question, and thus generates a time difference between a moment of time when the patient feels pain and a moment of time when the patient answers the examiner's question and requires an inconvenient procedure.

### SUMMARY OF THE INVENTION

Therefore, the present invention has been made in view of the above problems, and it is an object of the present invention to provide an auto examination system for intervertebral discs which measures an accurate point of time when a patient feels pain at an intervertebral disc by injecting a contrast material into the intervertebral disc at a regular velocity and then increasing internal pressure of the intervertebral disc from the outside.

It is a further object of the present invention to provide an auto examination system for intervertebral discs which uses data, obtained by synchronizing measured pressure data and a captured image of an intervertebral disc, as diagnosis data so as to more accurately confirm a point of time when a patient feels pain and thus to diagnose the intervertebral disc.

It is another object of the present invention to provide an auto examination system for intervertebral discs which uses data, obtained by synchronizing measured pressure data and a captured image of an intervertebral disc, as diagnosis data so as to investigate anatomic disorder in the intervertebral disc and a variation of internal pressure of the intervertebral disc, and factors influencing disc pain, thereby accurately investigating causes of disc pain.

It is yet another object of the present invention to provide an auto examination system for intervertebral discs which generates diagnosis data through synchronization of measured pressure data and a captured image of an intervertebral disc, and stores the generated diagnosis data for use in the future intervertebral disc diagnosis.

In accordance with the present invention, the above and other objects can be accomplished by the provision of an auto examination system for intervertebral discs including a contrast material injection device controlling a syringe filled with a contrast material to automatically adjust an injection velocity of the contrast material, and measuring pressure of an intervertebral disc of a patient, and a control device controlling the injection velocity of the contrast material according to information input by an examiner, and synchronizing an image supplied from an imaging device, seeing through bones and joints of a specific region of the patient, and pressure data of the intervertebral disc measured by the contrast material injection device to generate diagnosis data of the intervertebral disc and then to display and store the diagnosis data.

The contrast material injection device may include a contrast material injection pump controlling the syringe according to a control signal input from the control device to adjust the injection velocity of the contrast material, and transmitting information regarding an injection dose of the contrast material injected into the intervertebral disc to the control device, a digital pressure gauge measuring the pressure of the intervertebral disc, and a three-way valve interconnecting the syringe, a contrast material injection tube, and the digital pressure gauge.

The contrast material injection pump may be provided with a clamp to fix the syringe onto a mount, a slider to move a piston of the syringe, and spiral transfer shafts located between the mount and a holder to move the slider back and forth.

A contrast material injection balloon tube including an expansion member, which expands when the contrast material is injected thereinto, connected to one side thereof may be connected to the contrast material injection tube so as to examine an intervertebral disc of a patient, which has ruptured or is severely deformed and thus has no pressure difference when the contrast material is injected into the intervertebral disc.

The expansion member may be made of an elastic material so as to prevent the injected contrast material from leaking to the outside. The expansion member may be formed in a ring shape or an oval shape.

The contrast material injection device may include a power switch to control power supply, a fuse to interrupt power supply when high voltage/high current more than rated voltage is introduced, a power socket to be connected with a commercial AC power source, a serial transmission cable connector to transmit data to the control device, a ground terminal for grounding, and a pressure gauge connector to be connected with the digital pressure gauge.

Preferably, the contrast material injection device further includes a stop button to stop operation of the contrast material injection pump, and a warning light to visually indicate a warning state to the outside in the event of warning. More preferably, the contrast material injection device further includes a dose indicator to indicate a dose of the injected contrast material, and a pressure indicator to indicate a pressure state of the injected contrast material.

The digital pressure gauge may include a pressure sensor which receives a fluid, to which pressure is applied, introduced from a fluid supply tube, varies internal pressure thereof according to change of a diaphragm, and thus senses pressure. The pressure sensor may include a housing including the diaphragm to divide the inside thereof, a fluid introduction space provided at one side of the housing in communication with the fluid supply tube through a connection unit, an internal pressure variation space provided at the other side of the housing such that pressure in the internal pressure variation space is varied according to change of the diaphragm, and a pressure sensing device to sense a variation in the pressure in the internal pressure variation space and to convert the variation in the pressure into an electrical signal.

The connection unit may include a tap hole formed through one side wall of the fluid introduction space, and a connection cap connected with the fluid supply tube through a nipple-shaped digital pressure gauge connection tube, and coupled with the tap hole.

A plane of the diaphragm contacting the inner circumferential surface of the housing may be inclined or be stepped.

The connection cap may include a thin silicon plate to apply pressure to the diaphragm in proportion to the pressure of the introduced fluid.

The contrast material injection device may further include a switching mode power supply (SMPS) to convert commercial AC power supplied through the power switch into designated DC power, first to third DC-DC converters to convert the DC power supplied from the SMPS to designated DC voltage, a switch input circuit to generate an operation signal when the stop button is operated, an input detection circuit to detect an input signal, a central processing unit (CPU) to convert the pressure signal sensed by the pressure sensing device into data and then to transmit the data to the control device, to control the injection velocity of the contrast material under the control of the control device, and to control pressure and dose indication and to control driving of the warning light and a buzzer, a dose indication driving circuit to control driving of the dose indicator under the control of the CPU, a pressure indication driving circuit to control driving of the pressure indicator under the control of the CPU, a warning light turning-on circuit to control turning-on of the warning light under the control of the CPU, a buzzer driving circuit to drive the buzzer under the control of the CPU, a communication circuit to communicate with the control device under the control of the CPU, an external communication terminal connected with the communication circuit to communicate with the control device, a motor drive connected with the communication circuit to generate a motor drive control signal, and a motor driving circuit to drive a motor adjusting movement of the piston of the syringe injecting the contrast material according to the motor drive control signal generated from the motor drive.

The contrast material injection device may further include a motor drive forcible stopping circuit to generate a forcible stop control signal to forcibly stop the motor drive under the control of the CPU, a motor power control circuit to control drive power supplied to the motor drive according to the forcible stop control signal generated from the motor drive forcible stopping circuit, and a switching transistor connected to the motor power control circuit to supply or interrupt drive power to the motor drive.

The control device may include a data interface to interface with data of a key pad outputting an intensity of pain felt by the patient as an electrical signal through patient operation, voice data obtained by converting a patient's voice into an electrical signal, and pressure data generated from the contrast material injection device, an image interface to interface with image data generated from C-arm equipment, which sees through bones and joints of the patient and then displays an image of the bones and the joints, or image data of a digital camera capturing the image displayed on a monitor of the C-arm equipment, and a controller to generate and store intervertebral discs examination information through analysis of the pressure data, the pain data, and the voice data supplied from the data interface, to display disc diagnosis data, generated by synchronization of the image data supplied from the image interface and the pressure data, on a monitor, and to store the generated disc diagnosis data.

The controller may include an image CODEC unit to code the image of the C-arm equipment or the output image of the digital camera capturing the image displayed on the monitor of the C-arm equipment, supplied through the image interface, in an MPEG image processing format, a time synchronization signal generator to generate a time synchronization signal to synchronize the image processed by the image CODEC unit and the data, and a control unit to perform synchronization of the pressure data obtained from the data interface and the image processed by the image CODEC unit based on the time synchronization signal generated from the time synchronization signal generator, and to control display and storage of the synchronized image and data.

The control unit may include a setting module to set the auto examination system for intervertebral discs, a warning module to generate a warning when the auto examination system for intervertebral discs malfunctions, a control module to allow a user to control screen and data management and monitoring, an operating module to perform operation of the auto examination system for intervertebral discs, and a database to store information for intervertebral discs examination and data obtained as a result of the intervertebral disc examination.

The setting module may include a time setting function to stop the motor for a designated time, a velocity setting function to set the injection velocity of the contrast material, a dose setting function to set the injection dose of the contrast material, and an initial device pressure setting function to set an initial pressure of the contrast material injection device to 0 psi.

The control module may include a display function to regulate user's screen configuration and to display the information for intervertebral discs examination on the screen, a data management function to store various information about the patient, to read the stored data, and to manage data of peripheral devices, such as a printer, and a monitor function to monitor various velocities, dose, pressure, and pain data (Visual Analog Scale).

The operating module may include a start function to operate the contrast material injection device, a designated time temporary stop function to temporarily stop the contrast material injection device for a designated time, a temporary stop function to temporarily stop the contrast material injection device, and a stop function to completely stop the contrast material injection device.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view illustrating an auto examination system for intervertebral discs in accordance with the present invention;

FIG. 2 is a perspective view of a contrast material injection device of FIG. 1;

FIG. 3 is a plan view of the contrast material injection device of FIG. 2;

FIG. 4 is a bottom view of the contrast material injection device of FIG. 2;

FIGS. 5A to 5C are exemplary views illustrating an oval-shaped expansion member in use;

FIGS. 6A to 6C are exemplary views illustrating a ring-shaped expansion member in use;

FIGS. 7A and 7B are views illustrating an external appearance of a digital pressure gauge;

FIGS. 8 to 10 are cross-sectional views of pressure sensors in accordance with embodiments of the present invention;

FIG. 11 is a circuit diagram of the contrast material injection device and a control device in accordance with the present invention;

FIG. 12 is a block diagram of a controller within the control device of FIG. 11; and

FIG. 13 is a block diagram of the control unit of FIG. 12.

### DETAILED DESCRIPTION OF THE INVENTION

Now, a preferred embodiment of the present invention will be described in detail with reference to the annexed drawings. In the following description of the present invention, a detailed description of known functions and configurations incorporated herein will be omitted when it may make the subject matter of the present invention rather unclear.

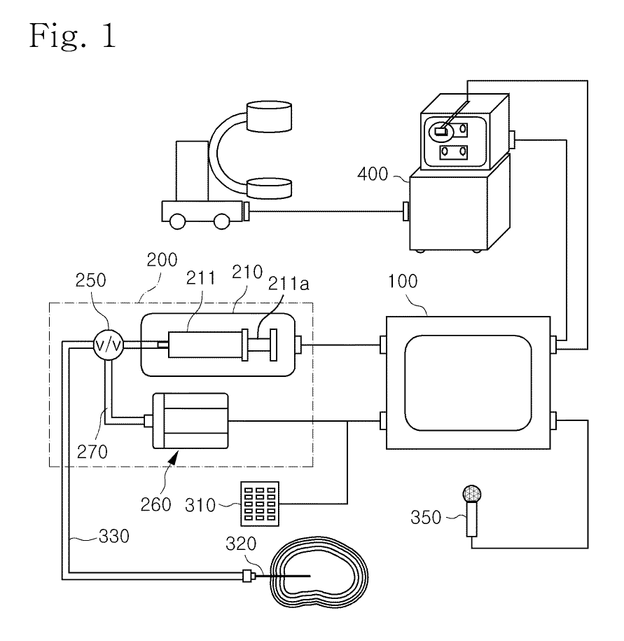

FIG. 1 is a schematic view illustrating an auto examination system for intervertebral discs in accordance with the present invention. The auto examination system for intervertebral discs includes a control device 100, a contrast material injection device 200, and C-arm equipment 400. Further, a needle 320 connected to the contrast material injection device 200 and injected into an intervertebral disc of a patient, a key pad 310 manipulated by the patient, and a microphone 350 to which a patient's voice is input are connected to the control device 100.

The C-arm equipment 400 which is a kind of X-ray equipment is an imaging apparatus to continuously see through bones and joints of a human body, and allows respective regions of the human body to be captured and developed and thus allows lesions to be accurately detected and be cured during nerve block for pain treatment or joint treatment. Such C-arm equipment 400 is widely used in medical institutions, such as hospitals, and a detailed description thereof will thus be omitted. Instead of the C-arm equipment 400, angiography equipment may be used.

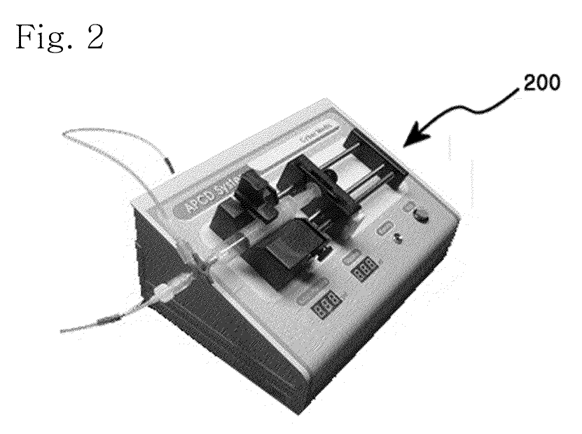

The contrast material injection device 200, as shown in FIG. 2, includes a contrast material injection pump 210, a digital pressure gauge 260 to measure pressure of a human intervertebral disc, and a three-way valve 250. The three-way valve 250 interconnects a syringe 211 of the contrast material injection pump 210, the digital pressure gauge 260, and the needle 320.

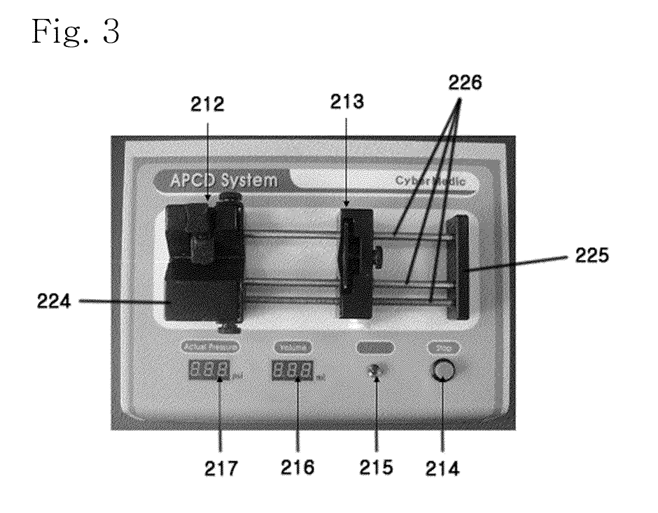

In the contrast material injection pump 210, as shown in FIG. 3, the syringe 211 is mounted in a V-shaped groove on a syringe mount 224, and a syringe clamp 212 to fix the syringe 211 mounted in the V-shaped groove is formed at one side of the mount 224.

A plurality of spiral transfer shafts 226 is installed between the syringe mount 224 to fix the syringe 211 and a holder 225, and a slider 213 to move a plunger 211a of the syringe 211 at a regular velocity is mounted along the spiral transfer shafts 226. The slider 213 is provided with through holes, to which the spiral transfer shafts 226 are inserted, so as to be guided horizontally. When a stepping motor (not shown) in the contrast material injection pump 210 is driven under the control of the control device 100 and thus rotates the spiral transfer shafts 226, the slider 213 moves along threads formed on the spiral transfer shafts 226 at a regular velocity and pushes the plunger 211a to inject the contrast material in the syringe 211 into an intervertebral disc of a patient at the regular velocity. Here, syringes having various shapes may be used as the syringe 211.

Through the three-way valve 250, the contrast material in the syringe 211 at one side is injected into the digital pressure gauge 260 via a fluid supply tube 270, and is injected into the intervertebral disc of the patient via the needle 320 connected to the other side of the three-way valve 250 by a contrast material injection tube 330.

The digital pressure gauge 260 receives data regarding an injection dose of the contrast material input from the control device 100, and controls the stepping motor to control a moving velocity of the plunger 211a of the syringe 211 based on the data, thereby adjusting an injection velocity of the contrast material.

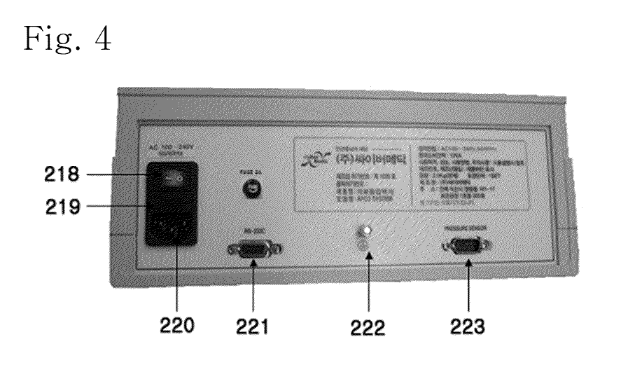

Further, the contrast material injection pump 210, as shown in FIGS. 3 and 4, includes a power switch 218 to control power supply from a power unit, a fuse 219 to interrupt power supply when high voltage/high current more than rated voltage is introduced, a power socket 220 to be connected with a commercial AC power source, a serial transmission cable connector 221 to transmit data to the control device 100, a ground terminal 222 for grounding, a pressure gauge connector 223 to be connected with the digital pressure gauge 260, a stop button 214 to stop operation of the contrast material injection pump 210, a warning light 215 to provide a visual warning in the event of warning, a dose indicator 216 to indicate a dose of the injected contrast material, and a pressure indicator 217 to indicate a pressure state of the injected contrast material.

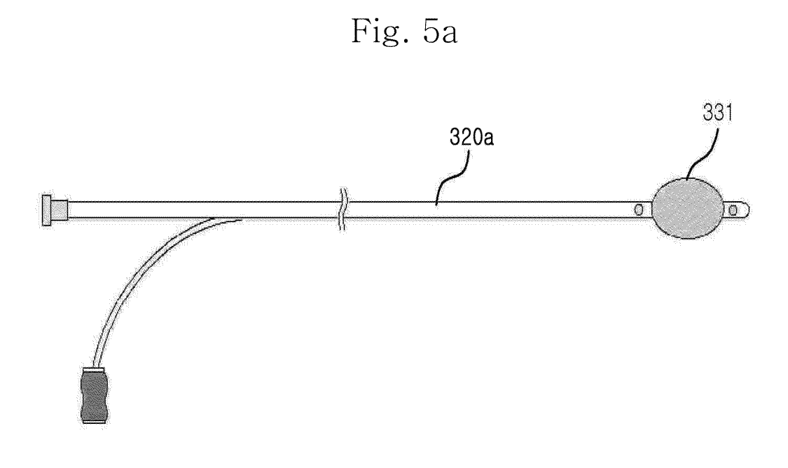

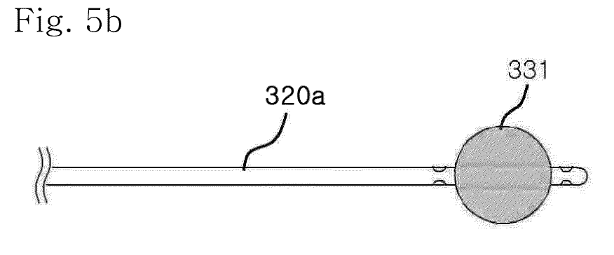

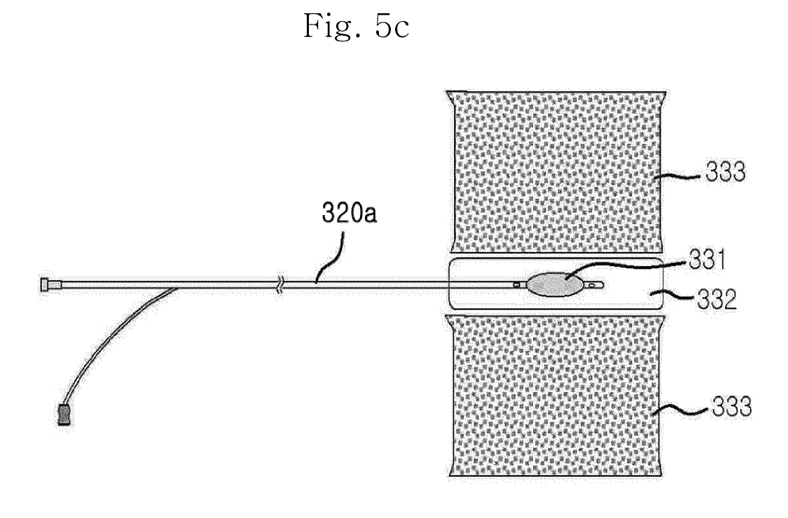

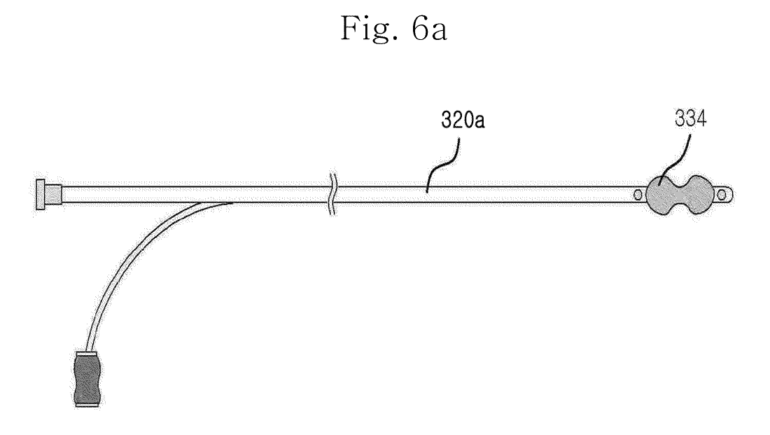

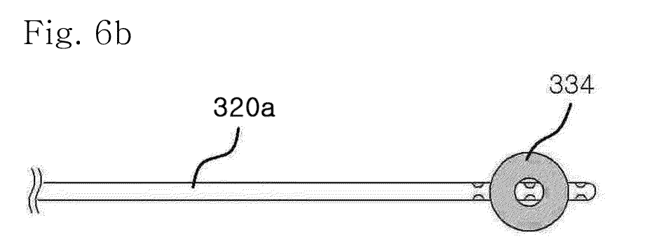

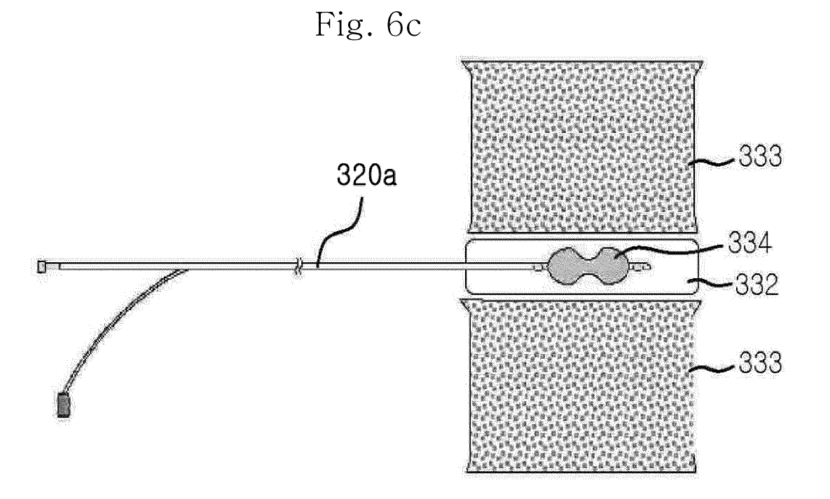

According to patient symptoms, instead of the needle 320, a contrast material injection balloon tube 320a, as shown in FIGS. 5A and 6A, may be used. That is, if an intervertebral disc 332 of a patient has ruptured, the injected contrast material leaks to the outside of the intervertebral disc 332, and thus there is no variation of internal pressure of the intervertebral disc 332 although the contrast material is injected into the intervertebral disc 332, the contrast material injection balloon tube 320a is used.

In order to examine an intervertebral disc of a patient in which there is no variation of internal pressure of the intervertebral disc even if the contrast material is injected into the intervertebral disc, the contrast material injection balloon tube 320a may include an expansion member 331 or 334, expanded when the contrast material is injected into the contrast material injection balloon tube 320a, at one side thereof. If there is no variation of the internal pressure of the intervertebral disc 332 due to leakage of the contrast material to the outside of the intervertebral disc 332 although the contrast material is injected into the intervertebral disc 332 by inserting the needle 320 into the intervertebral disc 332, the needle 320 is taken out of the intervertebral disc 332, and then the contrast material injection balloon tube 320a, as shown in FIG. 5C, is injected into the intervertebral disc 332. Thereafter, when the contrast material is injected into the intervertebral disc 332 through the contrast material injection balloon tube 320a, the oval-shaped expansion member 332, which is formed at the tip of the contrast material injection balloon tube 320a and is made of a soft elastic material to prevent the contrast material from leaking to the outside of the intervertebral disc 332, is expanded, as shown in FIG. 5B, and thus applies pressure to the inside of the intervertebral disc 332. Thereby, even if the intervertebral disc 332 has ruptured, it is possible to measure the internal pressure of the intervertebral disc 332.

FIGS. 6A to 6C illustrate the ring-shaped expansion member 334 modified from the above oval-shaped expansion member 331. These shapes of the expansion member are only examples, and thus those skilled in the art will appreciate that various modifications of the shape of the expansion member are possible.

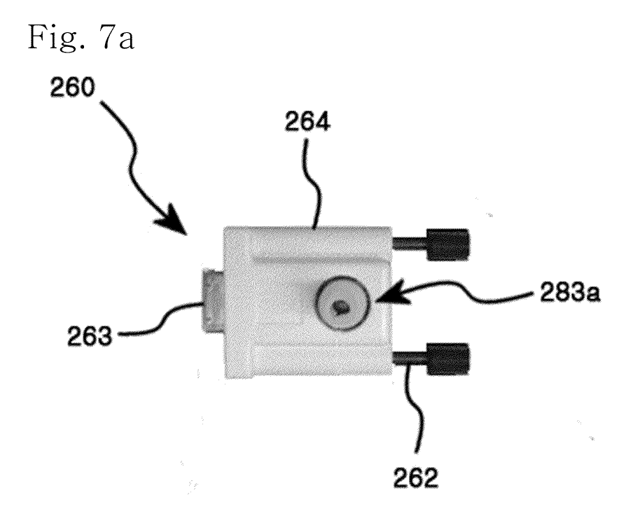

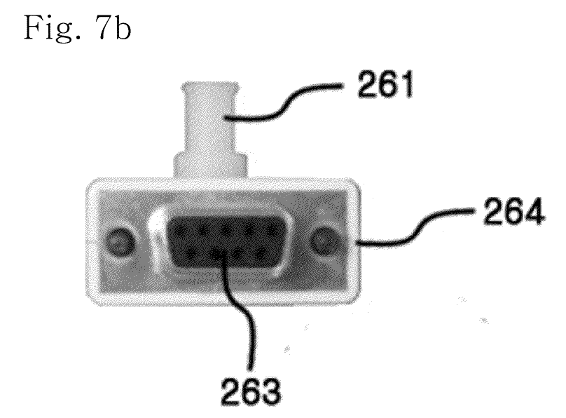

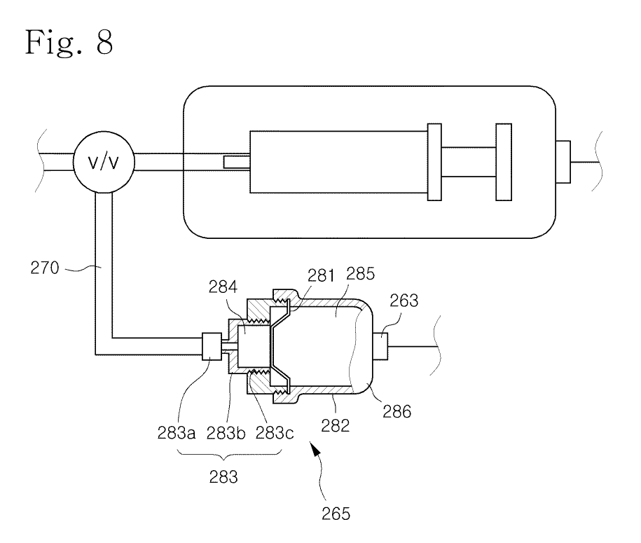

The digital pressure gauge 260 is a device to detect internal pressure of an intervertebral disc of a patient. The digital pressure gauge 260, as shown in FIGS. 7A and 7B, includes a main body 264, a digital pressure gauge connection tube 283a and a digital pressure gauge fixing terminal 262 provided on the main body 264, and a data connection port 263 provided at one side of the main body 264 to transmit measured pressure data to the control device 100. As shown in FIGS. 8 to 10, a pressure sensor 265 to sense pressure according fluid pressure of the contrast material introduced from the fluid supply tube 270 through the digital pressure gauge connection tube 283a is provided in the digital pressure gauge main body 264. The pressure sensor 265 includes a housing 282 including a diaphragm 281 to divide the inside thereof, a fluid introduction space 284 provided at one side of the housing 282 in communication with the fluid supply tube 270 through the digital pressure gauge connection tube 283a, an internal pressure variation space 285 provided at the other side of the housing 282 such that internal pressure of the internal pressure variation space 285 is varied according to change of the diaphragm 281, and a pressure sensing device 286 to sense a variation in the pressure in the internal pressure variation space 285 and to convert the variation in the pressure into an electrical signal.

Here, a connection unit 283 includes a tap hole 283c formed through one side wall of the fluid introduction space 284, and a connection cap 283b connected with the fluid supply tube 270 through the nipple-shaped digital pressure gauge connection tube 283a and coupled with the tap hole 283c.

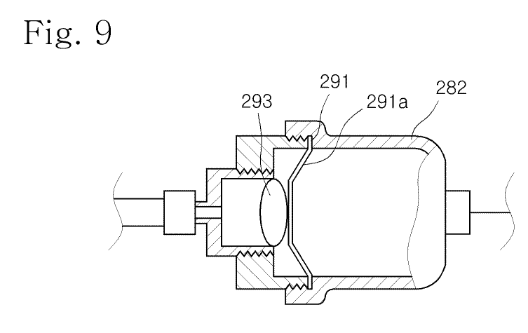

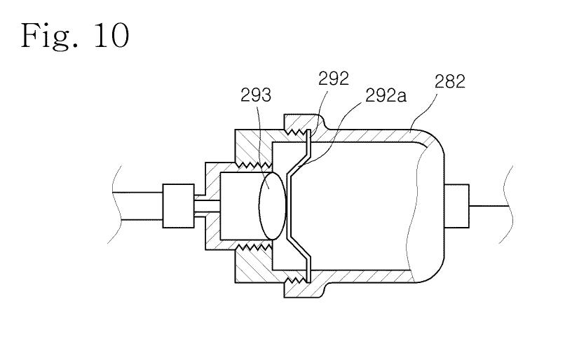

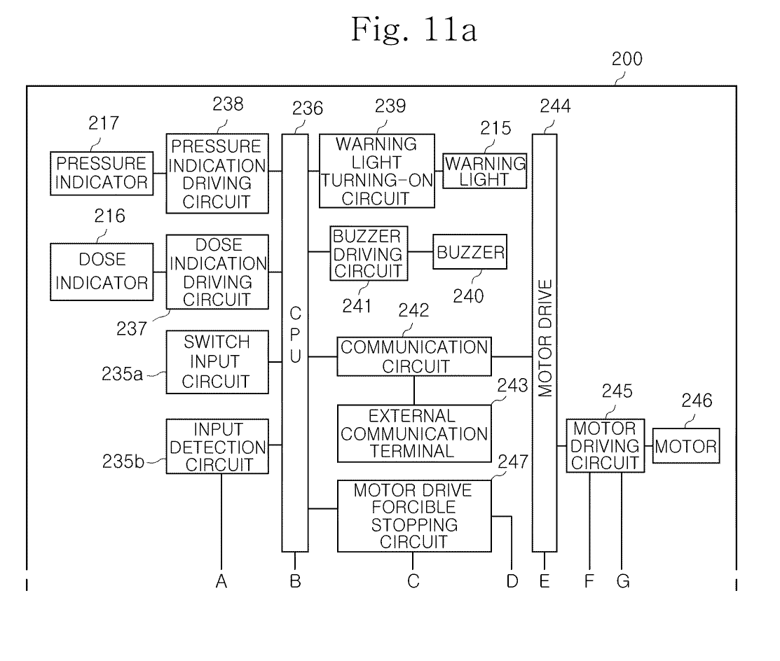

FIGS. 9 and 10 respectively illustrate diaphragms 291 and 292 in accordance with other embodiments of the present inventions. As shown in FIG. 9, the diaphragm 291 includes an inclined plane 291a at a portion thereof contacting the inner circumferential surface of the housing 282, and as shown in FIG. 10, the diaphragm 292 includes a stepped part 292a at a portion thereof contacting the inner circumferential surface of the housing 282.

Further, a thin silicon plate 293 to apply pressure to the diaphragm 281, 291, or 292 in proportion to the pressure of a fluid introduced to the fluid introduction space 284 may be provided within the fluid introduction space 284.

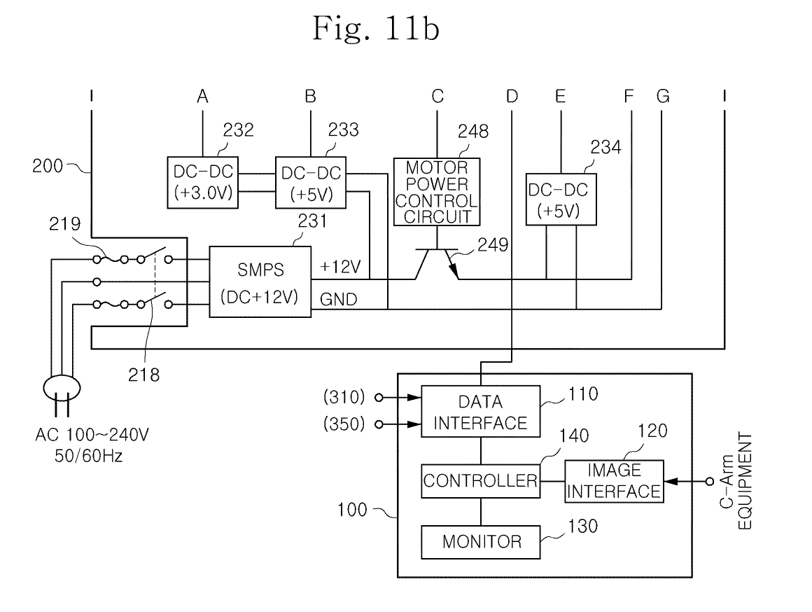

As shown in FIG. 11, the contrast material injection pump 210 includes a switching mode power supply (SMPS) 231 to convert commercial AC power supplied through the power switch 218 into designated DC power, first to third DC-DC converters 23˜234 to convert the DC power supplied from the SMPS 231 to designated DC voltage, a switch input circuit 235a to generate an operation signal when the stop button is operated, an input detection circuit 235b to detect an input signal, a central processing unit (CPU) 236 to convert the pressure signal sensed by the pressure sensing device 286 into data and then to transmit the data to the control device 100, to control an injection velocity of the contrast material under the control of the control device 100, and to control pressure and dose indication and to control driving of the warning light 215 and a buzzer 240, a dose indication driving circuit 237 to control driving of the dose indicator 216 under the control of the CPU 236, a pressure indication driving circuit 239 to control driving of the pressure indicator 217 under the control of the CPU 236, a warning light turning-on circuit 239 to control turning-on of the warning light 215 under the control of the CPU 236, a buzzer driving circuit 241 to drive the buzzer 240 under the control of the CPU 236, a communication circuit 242 to communicate with the control device 100 under the control of the CPU 236, an external communication terminal 243 connected with the communication circuit 242 to communicate with the control device 100, a motor drive 244 connected with the communication circuit 242 to generate a motor drive control signal, a motor driving circuit 245 to drive a motor 246 adjusting movement of the plunger 211a of the syringe 211 injecting the contrast material according to the motor drive control signal generated from the motor drive 244, a motor drive forcible stopping circuit 247 to generate a forcible stop control signal to forcibly stop the motor drive 244 under the control of the CPU 236, a motor power control circuit 248 to control drive power supplied to the motor drive 244 according to the forcible stop control signal generated from the motor drive forcible stopping circuit 247, and a switching transistor 249 connected to the motor power control circuit 248 to supply or interrupt drive power to the motor drive 244.

Further, the control device 100, as shown in FIG. 11, includes a data interface 110 to interface with data of the key pad 310 outputting an intensity of pain felt by a patient as an electrical signal through patient operation, voice data obtained by converting the patient's voice into an electrical signal through the microphone 350, and the pressure data generated from the contrast material injection device 200, an image interface 120 to interface with image data generated from the C-arm equipment 400, which sees through bones and joints of the patient and then displays an image of the bones and the joints, and a controller 140 to generate and store information for intervertebral discs examination through analysis of the pressure data, the pain data, and the voice data supplied from the data interface 110, to display disc diagnosis data, generated by synchronization of the image data supplied from the image interface 120 and the pressure data, on a monitor 130, and to store the generated disc diagnosis data.

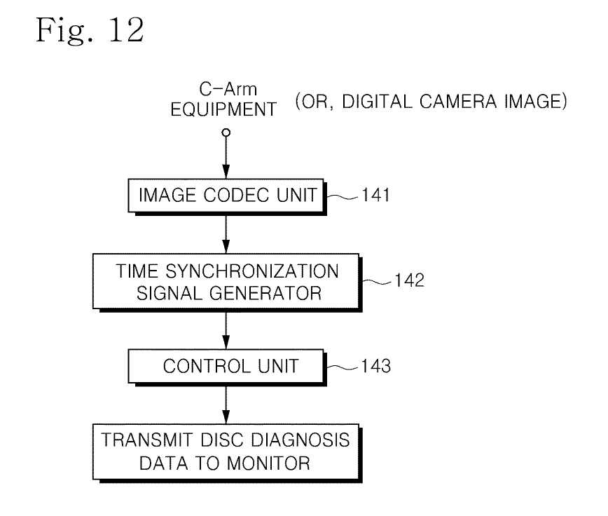

The controller 140, as shown in FIG. 12, includes an image CODEC unit 141 to code the image of the C-arm equipment 400 supplied through the image interface 120 or an output image of a digital camera (or a CCTV) capturing the image displayed on a monitor of the C-arm equipment 400 in an MPEG image processing format, a time synchronization signal generator 142 to generate a time synchronization signal to synchronize the image processed by the image CODEC unit 141 and the data, and a control unit 143 to perform synchronization of the pressure data obtained from the data interface 110 and the image processed by the image CODEC unit 141 based on the time synchronization signal generated from the time synchronization signal generator 142, and to control display and storage of the synchronized image and data.

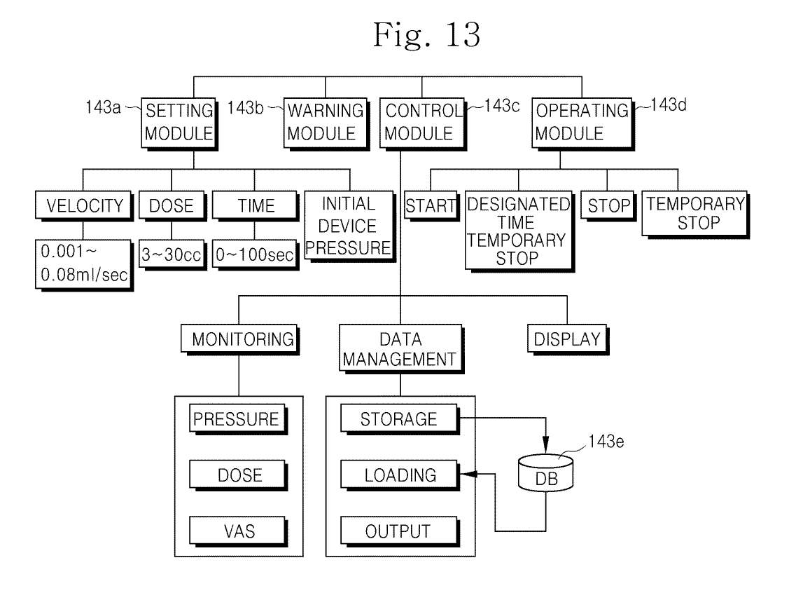

Here, the control unit 143, as shown in FIG. 13, includes a setting module 143a to set the auto examination system for intervertebral discs, a warning module 143b to generate a warning when the auto examination system for intervertebral discs malfunctions, a control module 143c to allow a user to control screen and data management and monitoring, an operating module 143d to perform operation of the auto examination system for intervertebral discs, and a database 143e to store information for intervertebral discs examination and data obtained as a result of the intervertebral disc examination.

The setting module 143a includes a time setting function to stop the motor 246 for a designated time, a velocity setting function to set an injection velocity of the contrast material, a dose setting function to set an injection dose of the contrast material, and an initial device pressure setting function to set an initial pressure of the contrast material injection device 200 to 0 psi.

The control module 143c includes a display function to regulate user's screen configuration and to display the information for intervertebral discs examination on the screen, a data management function to store various information about the patient, to read the stored data, and to manage data of peripheral devices, such as a printer, and a monitor function to monitor various velocities, dose, pressure, and pain data (Visual Analog Scale).

The operating module 143d includes a start function to operate the contrast material injection device 200, a designated time temporary stop function to temporarily stop the contrast material injection device 200 for a designated time, a temporary stop function to temporarily stop the contrast material injection device 200, and a stop function to completely stop the contrast material injection device 200.

Hereinafter, a method of operating the above auto examination system for intervertebral discs in accordance with the present invention will be described, as follows.

First, an operator mounts the syringe 211 filled with the contrast material on the contrast material injection pump 210 of the contrast material injection device 200, and fixes the syringe 211 by adjusting the syringe clamp 212. Thereafter, the front end of the syringe 211 is connected to the three-way valve 250, and then the contrast material injection tube 330 and the digital pressure gauge 260 are connected to the three-way valve 250. Here, the connection order of the syringe 211, the contrast material injection tube 330, and the digital pressure gauge 260 to the three-way valve 250 may be carried out freely.

Under the condition that the contrast material injection device 200 and the control device 100 are connected and the C-arm equipment 400 and the control device 100 are connected, the C-arm equipment 400 is located at a set position of a patient and the pressure of the digital pressure gauge 260 is initialized to 0 psi. The initialization of the pressure of the digital pressure gauge 260 to 0 psi is carried out by opening the three-way valve 250 such that air in a connection line is discharged. Thereafter, the three-way valve 250 is closed such that the contrast material is introduced in the direction of the digital pressure gauge 260 while the air in the connection line of the digital pressure gauge 260, i.e., the fluid supply tube 270, is discharged.

Thereafter, when the operator (an examiner) inputs a diameter and an injection velocity of the syringe 211 through an input device (not shown), the corresponding data are input to the controller 140 of the control device 100.

The controller 140 stores the diameter and the injection velocity of the syringe 211, generates injection velocity control data, and transmits the generated data to the CPU 236 of the contrast material injection device 200. The CPU 236 controls the motor drive 244 corresponding to the transmitted injection velocity control data so as to drive the motor 246, and when the spiral transfer shafts 226 provided with threads formed thereon are rotated by the driving of the motor 246, the slider 213 moves forward in the direction of the mount 224 to move the plunger 211a of the syringe 211 at a regular velocity and thus to cause the contrast material to start to be injected into an intervertebral disc of the patient. Here, the needle 320 is assumed to have been inserted into the set position of the intervertebral disc of the patient.

When the contrast material starts to be injected into the intervertebral disc of the patient, the CPU 236 transmits the volume (dose) of the injected contrast material to the control device 100 in real time, and simultaneously controls the dose indication driving circuit 237 to indicate the injection dose though the dose indicator 216.

From this time, the control device 100 starts to collect intervertebral disc examination results.

When the intervertebral disc examination starts to be performed, the control device 100 receives, processes, and stores the injection dose of the contrast material output from the contrast material injection pump 210, the voice signal output from the microphone 350, the pressure data output from the digital pressure gauge 260, and the pain data output from the key pad 310, synchronizes the processed pressure data and the captured image transmitted from the C-arm equipment 400, and displays disc diagnosis data, obtained by the synchronization, on the monitor 130.

One feature of the auto examination system for intervertebral discs in accordance with the present invention is that diagnosis data is generated by synchronizing the pressure data obtained by the digital pressure gauge 260 and the capture image obtained by the C-arm equipment 400, is stored, and is used as pain diagnosis data in intervertebral disc diagnosis. A further feature of the auto examination system for intervertebral discs in accordance with the present invention is that the pressure data and the captured image generating the current pressure are simultaneously displayed on a single screen on the monitor 130 so as to allow the examiner to accurately check up the intervertebral disc.

The synchronization of the pressure data and the captured data will be described in detail later.

The controller 140 of the control device 100 converts voltage output from the digital pressure gauge 260 into a pressure value using a calculation expression, converts text information of the injection dose of the contrast material into a numeral, converts the voice signal output from the microphone 350 into a voice signal in a dB scale, and converts the pain data, consisting of a voltage value, output from the key pad 310 into a numeral in the range of 0˜10, and then processes the input data.

Such an input data processing procedure of the controller 140 employs a well-known method used in conventional examination systems for intervertebral discs, and a detailed description thereof will thus be omitted.

Further, after the control device 100 processes the pressure data input during the process of performing the examination for intervertebral discs, the control device 100 transmits the pressure data to the CPU 236 of the contrast material injection device 200 so as to indicate the pressure data, and the CPU 236 controls the pressure indication driving circuit 238 such that the pressure indicator 217 indicates the current pressure of the intervertebral disc of the patient in real time.

Further, the control device 100 generates pressure data through analysis of the pressure data and the injection dose of the contrast material, and, if the pressure exceeds 120 psi or the injection dose of the contrast material exceeds 3 cc, the control device 100 generates warning data and transmits the warning data to the CPU 236, the CPU 236 drives the buzzer 240 through the buzzer driving circuit 241 and turns on the warning light 215 through the warning light turning-on circuit 239, thereby allowing the examiner to accurately recognize the current state of the auto examination system for intervertebral discs.

Of course, if an excessively large amount of the contrast amount is injected into the intervertebral disc of the patient and thus the pressure of the intervertebral disc is more than a regulated value or the auto examination system for intervertebral discs malfunctions, the control device 100 automatically transmits command data to stop the injection of the contrast material to the CPU 236 of the contrast material injection device 200, and the CPU 236 drives the motor power control circuit 248 through the motor drive forcible stop circuit 247. The switching transistor 249 is turned off by the driving of the motor power driving circuit 248, and thus interrupts power supplied to the third DC-DC converter 234. Thereby, the operation of the motor drive 244 and the operation of the motor driving circuit 245 are stopped.

Further, another feature of the auto examination system for intervertebral discs in accordance with the present invention is that the digital pressure gauge 260 accurately detects the pressure of the patient's intervertebral disc.

That is, the digital pressure gauge 260 is a device to detect the pressure of the intervertebral disc of the patient, and includes the main body 264, and the digital pressure gauge connection tube 283a and the digital pressure gauge fixing terminal 262 provided on the main body 264, as shown in FIGS. 7A and 7B.

Further, the data connection port 263 to transmit measured pressure data to the control device 100 is provided at one side of the main body 264, thus transmitting the pressure data to the control device 100.

Such a digital pressure gauge 260 is provided with the pressure sensor 265, as shown in FIG. 8. When introduction of the contrast material is started, the contrast material, to which pressure is applied, is introduced from the fluid supply tube 270 connected with the three-way valve 250, and then the contrast material introduced through the nipple-shaped digital pressure gauge connection tube 283a presses the diaphragm 281. Here, the diaphragm 281 is changed according to the pressure of the contrast material, the pressure of the internal pressure variation space 285 is varied according to the change of the diaphragm 281, and the pressure sensing device 286 senses the variation in the pressure of the internal pressure variation space 285, converts the variation in the pressure into an electrical signal, and provides the signal as pressure data to the control device 100 through the data connection port 263.

Here, the diaphragm 281 may be modified so as to have various shapes.

For example, as shown in FIG. 9, the diaphragm 291 may include the inclined plane 291a at a portion thereof contacting the inner circumferential surface of the housing 282, and as shown in FIG. 10, the diaphragm 292 may include the stepped part 292a at a portion thereof contacting the inner circumferential surface of the housing 282. Further, the thin silicon plate 293 to apply pressure to the diaphragm 281, 291, or 292 in proportion to the pressure of a fluid introduced to the fluid introduction space 284 may be provided within the fluid introduction space 284.

Although the above digital pressure gauge 260 may accurately detect pressure of a human intervertebral disc in real time, the needle 320 may be replaced with the expansion member 331 or 334, as shown in FIG. 5A or 6A, to precisely measure pressure of a human intervertebral disc according to patient's conditions. If an intervertebral disc of a patient has considerably ruptured and the injected contrast material leaks to the outside of the intervertebral disc, the expansion member 331 or 334 is used. That is, when there is no pressure detected although the contrast material is injected into an intervertebral disc of a patient, it is judged that the contrast material leaks due to intervertebral disc rupture.

In this case, the initially inserted needle 320 is taken out of the intervertebral disc 332, and then the contrast material injection balloon tube 320a, as shown in FIG. 5C, is injected into the intervertebral disc 332. Thereafter, when the contrast material is injected into the intervertebral disc 332 through the contrast material injection balloon tube 320a, the oval-shaped expansion member 332, which is formed at the tip of the contrast material injection balloon tube 320a and is made of a soft elastic material to prevent the contrast material from leaking to the outside of the intervertebral disc 332, is expanded, as shown in FIG. 5B, and thus applies pressure to the intervertebral disc 332. Thereby, although the intervertebral disc 332 has ruptured, it is possible to measure pressure of the intervertebral disc 332.

Further, the control device 100 generates intervertebral disc examination information using the injection dose of the contrast material, the voice signal, the pressure data, the pain data, and the captured intervertebral disc image, processed in real time, and then displays the intervertebral discs examination information on the monitor 130.

For this purpose, in the controller 140, as shown in FIG. 12, the image CODEC unit 141 codes the image of the C-arm equipment 400 supplied through the image interface 120 or the output image of the digital camera capturing the image displayed on the monitor of the C-arm equipment 400 in the MPEG image processing format, and the time synchronization signal generator 142 generates a time synchronization signal to synchronize the image processed by the image CODEC unit 141 and the data.

The control unit 143 of the controller 140 performs synchronization of the pressure data obtained from the data interface 110 and the image processed by the image CODEC unit 141 based on the time synchronization signal generated from the time synchronization signal generator 142, controls display of the synchronized image and data, and stores the synchronized image and data in the database 143e. Here, since all C-arm equipment 400 is not provided with an image output terminal, a digital camera may be employed to use image data of the C-arm equipment 400 without an image output terminal. For example, the digital camera captures the monitor of the C-arm equipment 400 to generate image data, and then codes the image data into an MPEG image through the image interface 120.

In the control unit 143, as shown in FIG. 13, the setting module 143a performs the time setting function to stop the motor 246 for a designated time, the velocity setting function to set an injection velocity of the contrast material, the dose setting function to set an injection dose of the contrast material, and the initial device pressure setting function to set an initial pressure of the contrast material injection device 200 to 0 psi.

Further, the control module 143c performs the display function to regulate user's screen configuration and to display the information for intervertebral discs examination on the screen, the data management function to store various information about the patient, to read the stored data, and to manage data of peripheral devices, such as a printer, and the monitor function to monitor various velocities, dose, pressure, and pain data (VAS).

Further, the operating module 143d performs the start function to operate the contrast material injection device 200, the designated time temporary stop function to temporarily stop the contrast material injection device 200 for a designated time, the temporary stop function to temporarily stop the contrast material injection device 200, and the stop function to completely stop the contrast material injection device 200.

Further, the intervertebral discs examination information formed by the control device 100 may include a graph illustrating relations between the injection dose of the contrast material and the internal pressure of the intervertebral disc, a real time pressure chart, a pressure gauge, a performance number, the injection dose of the contrast material, voice data (may be expressed in a graph in a dB scale), a static pressure, a pressure difference, pain data, etc.

As apparent from the above description, the present invention provides an automatic examination system for intervertebral discs which injects a contrast material into the intervertebral disc at a regular velocity, and accurately measures internal pressure of the intervertebral disc from the outside.

Further, the auto examination system for intervertebral discs uses data, obtained by synchronizing measured pressure data and a captured image of the intervertebral disc, as diagnosis data, thereby more accurately diagnosing the intervertebral disc.

Further, the auto examination system for intervertebral discs uses data, obtained by synchronizing the measured pressure data and the captured image of the intervertebral disc, as diagnosis data, thereby accurately investigating causes of lumbago in discs.

Moreover, the auto examination system for intervertebral discs generates the diagnosis data through synchronization of the measured pressure data and the captured image of the intervertebral disc, and stores the generated diagnosis data, thereby enabling use of the stored diagnosis data for the future intervertebral disc diagnosis.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. An auto examination system for intervertebral discs comprising:
a contrast material injection device controlling a syringe filled with a contrast material to automatically adjust an injection velocity of the contrast material, and measuring pressure of an intervertebral disc of a patient; and
a control device controlling the injection velocity of the contrast material according to information input by an examiner, and synchronizing an image supplied from an imaging device, seeing through bones and joints of a specific region of the patient, and pressure data of the intervertebral disc measured by the contrast material injection device to generate diagnosis data of the intervertebral disc and then to display and store the diagnosis data.

2. The auto examination system for intervertebral discs according to claim 1, wherein the imaging device is C-arm equipment or angiography equipment.

3. The auto examination system for intervertebral discs according to claim 1, wherein the contrast material injection device includes:
a contrast material injection pump controlling the syringe according to a control signal input from the control device to adjust the injection velocity of the contrast material, and transmitting information regarding an injection dose of the contrast material injected into the intervertebral disc to the control device;
a digital pressure gauge measuring the pressure of the intervertebral disc; and
a three-way valve interconnecting the syringe, a contrast material injection tube, and the digital pressure gauge.

4. The auto examination system for intervertebral discs according to claim 3, wherein the contrast material injection pump is provided with:
a clamp to fix the syringe onto a mount;
a slider to move a piston of the syringe; and
spiral transfer shafts located between the mount and a holder to move the slider back and forth.

5. The auto examination system for intervertebral discs according to claim 3, wherein a contrast material injection balloon tube including an expansion member, which expands when the contrast material is injected thereinto, connected to one side thereof is connected to the contrast material injection tube so as to examine an intervertebral disc of a patient, which has ruptured or is severely deformed and thus has no pressure difference when the contrast material is injected into the intervertebral disc.

6. The auto examination system for intervertebral discs according to claim 5, wherein the expansion member is made of an elastic material so as to prevent the injected contrast material from leaking to the outside and is formed in a ring shape or an oval shape.

7. The auto examination system for intervertebral discs according to claim 3, wherein the contrast material injection device includes:
a power switch to control power supply;
a fuse to interrupt power supply when high voltage/high current more than rated voltage is introduced;
a power socket to be connected with a commercial AC power source;
a serial transmission cable connector to transmit data to the control device;
a ground terminal for grounding; and
a pressure gauge connector to be connected with the digital pressure gauge.

8. The auto examination system for intervertebral discs according to claim 7, wherein the contrast material injection device further includes:
a stop button to stop operation of the contrast material injection pump; and
a warning light to visually indicate a warning state to the outside in the event of warning.

9. The auto examination system for intervertebral discs according to claim 7, wherein the contrast material injection device further includes:
a dose indicator to indicate a dose of the injected contrast material; and
a pressure indicator to indicate a pressure state of the injected contrast material.

10. The auto examination system for intervertebral discs according to claim 3, wherein the digital pressure gauge includes a pressure sensor which receives a fluid, to which pressure is applied, introduced from a fluid supply tube, varies internal pressure thereof according to change of a diaphragm, and thus senses pressure.

11. The auto examination system for intervertebral discs according to claim 10, wherein the pressure sensor includes:
a housing including the diaphragm to divide the inside thereof;
a fluid introduction space provided at one side of the housing in communication with the fluid supply tube through a connection unit;
an internal pressure variation space provided at the other side of the housing such that pressure within the internal pressure variation space is varied according to change of the diaphragm; and
a pressure sensing device to sense a variation in the pressure in the internal pressure variation space and to convert the variation in the pressure into an electrical signal.

12. The auto examination system for intervertebral discs according to claim 11, wherein the connection unit includes:
a tap hole formed through one side wall of the fluid introduction space; and
a connection cap connected with the fluid supply tube through a nipple-shaped digital pressure gauge connection tube, and coupled with the tap hole.

13. The auto examination system for intervertebral discs according to claim 11, wherein a plane of the diaphragm contacting the inner circumferential surface of the housing is inclined.

14. The auto examination system for intervertebral discs according to claim 12, wherein the connection cap includes a thin silicon plate to apply pressure to the diaphragm in proportion to the pressure of the introduced fluid.

15. The auto examination system for intervertebral discs according to claim 9, wherein the contrast material injection device further includes:
a switching mode power supply (SMPS) to convert commercial AC power supplied through the power switch into designated DC power;
first to third DC-DC converters to convert the DC power supplied from the SMPS to designated DC voltage;
a switch input circuit to generate an operation signal when the stop button is operated;
an input detection circuit to detect an input signal;
a central processing unit (CPU) to convert the pressure signal sensed by the pressure sensing device into data and then to transmit the data to the control device, to control the injection velocity of the contrast material under the control of the control device, and to control pressure and dose indication and to control driving of the warning light and a buzzer;
a dose indication driving circuit to control driving of the dose indicator under the control of the CPU;
a pressure indication driving circuit to control driving of the pressure indicator under the control of the CPU;
a warning light turning-on circuit to control turning-on of the warning light under the control of the CPU;
a buzzer driving circuit to drive the buzzer under the control of the CPU;
a communication circuit to communicate with the control device under the control of the CPU;
an external communication terminal connected with the communication circuit to communicate with the control device;
a motor drive connected with the communication circuit to generate a motor drive control signal; and
a motor driving circuit to drive a motor adjusting movement of the piston of the syringe injecting the contrast material according to the motor drive control signal generated from the motor drive.

16. The auto examination system for intervertebral discs according to claim 15, wherein the contrast material injection device further includes:
a motor drive forcible stopping circuit to generate a forcible stop control signal to forcibly stop the motor drive under the control of the CPU;
a motor power control circuit to control drive power supplied to the motor drive according to the forcible stop control signal generated from the motor drive forcible stopping circuit; and
a switching transistor connected to the motor power control circuit to supply or interrupt drive power to the motor drive.

17. The auto examination system for intervertebral discs according to claim 1, further comprising a key pad outputting an intensity of pain felt by the patient as an electrical signal; and
a microphone converting a patient's voice into an electrical signal and then outputting the electrical signal.

18. The auto examination system for intervertebral discs according to claim 1, wherein the control device includes:
a data interface to interface with data of a key pad outputting an intensity of pain felt by the patient as an electrical signal through patient operation, voice data obtained by converting a patient's voice into an electrical signal, and pressure data generated from the contrast material injection device;
an image interface to interface with image data generated from C-arm equipment, which sees through bones and joints of the patient and then displays an image of the bones and the joints, or image data of a digital camera capturing the image displayed on a monitor of the C-arm equipment; and
a controller to generate and store intervertebral discs examination information through analysis of the pressure data, the pain data, and the voice data supplied from the data interface, to display disc diagnosis data, generated by synchronization of the image data supplied from the image interface and the pressure data, on a monitor, and to store the generated disc diagnosis data.

19. The auto examination system for intervertebral discs according to claim 18, wherein the disc diagnosis data displayed on the monitor includes a pressure graph, the pressure data, the injection dose of the contrast material, and the image data of the intervertebral disc of the patient from the C-arm equipment.

20. The auto examination system for intervertebral discs according to claim 18, wherein the controller includes:
an image CODEC unit to code the image of the C-arm equipment or the output image of the digital camera capturing the image displayed on the monitor of the C-arm equipment, supplied through the image interface, in an MPEG image processing format;
a time synchronization signal generator to generate a time synchronization signal to synchronize the image processed by the image CODEC unit and the data; and
a control unit to perform synchronization of the pressure data obtained from the data interface and the image processed by the image CODEC unit based on the time synchronization signal generated from the time synchronization signal generator, and to control display and storage of the synchronized image and data.

21. The auto examination system for intervertebral discs according to claim 20, wherein the control unit includes:
a setting module to set the auto examination system for intervertebral discs;
a warning module to generate a warning when the auto examination system for intervertebral discs malfunctions;
a control module to allow a user to control screen and data management and monitoring;
an operating module to perform operation of the auto examination system for intervertebral discs; and
a database to store information for intervertebral discs examination and data obtained as a result of the intervertebral disc examination.

22. The auto examination system for intervertebral discs according to claim 21, wherein the setting module includes:
a time setting function to stop the motor for a designated time;
a velocity setting function to set the injection velocity of the contrast material;
a dose setting function to set the injection dose of the contrast material; and
an initial device pressure setting function to set an initial pressure of the contrast material injection device to 0 psi.

23. The auto examination system for intervertebral discs according to claim 21, wherein the control module includes:
a display function to regulate user's screen configuration and to display the information for intervertebral discs examination on the screen;
a data management function to store various information about the patient, to read the stored data, and to manage data of peripheral devices, such as a printer; and
a monitor function to monitor various velocities, dose, pressure, and pain data (VAS).

24. The auto examination system for intervertebral discs according to claim 21, wherein the operating module includes:
a start function to operate the contrast material injection device;
a designated time temporary stop function to temporarily stop the contrast material injection device for a designated time;
a temporary stop function to temporarily stop the contrast material injection device; and
a stop function to completely stop the contrast material injection device.
